(19) United States
(12) Patent Application Publication
(10) Pub. No.: US 2026/0283834 A1
(43) Pub. Date: Sep. 24, 2026

(54) WEARABLE DEVICE FOR OSTOMY LEAKAGE DETECTION SYSTEM

(71) Applicant: Hollister Incorporated, Libertyville, IL (US)

(72) Inventors: Jonas P. Carlsson, Chicago, IL (US); Lauren M. Lattanzi, Raleigh, NC (US); Anthony B. Smith, Durham, NC (US); Grace E. Rhinehart, Cary, NC (US)

(73) Assignee: Hollister Incorporated, Libertyville, IL (US)

(21) Appl. No.: 19/472,971

(22) PCT Filed: May 24, 2024
(86) PCT No.: PCT/US2024/031091

§ 371 Date: Oct. 6, 2025

Related U.S. Application Data
(60) Provisional application No. 63/510,652, filed on Jun. 28, 2023.

(51) Int. Cl. A61F 5/44

(52) U.S. Cl. CPC A61F 5/4404

(57) ABSTRACT

An ostomy leakage detection system can include a wearable device and an adhesive patch. The wearable device may be configured to connect to a sensing accessory for detecting a leak. The wearable device may include a top housing, a bottom housing, and a hinge. The bottom housing may include a release layer. The adhesive patch may be configured to mount the wearable device. The adhesive patch may include a double-sided adhesive. The adhesive patch may include a first side configured to attach to a user and a second side configured to attach to the wearable device at the release layer.

### BACKGROUND

This disclosure is related to a leakage detection system for medical devices, and more particularly to a wearable device for an ostomy leakage detection system.

Known ostomy pouch systems can include a pouch formed from opposing sidewalls defining an internal collection area, an inlet opening for receiving a stoma, and an ostomy appliance for attaching the pouch to a user. Commonly, such ostomy appliance may include, for example, an ostomy barrier of a one-piece pouch system which can be attached to one of the pouch sidewalls proximate an inlet opening, or a faceplate for a two-piece pouch system configured to releasably engage a pouch, and a barrier ring. The ostomy appliance may include a skin barrier material for adhering to and sealing against user's peristomal skin surrounding the stoma.

However, the ostomy appliance may be susceptible to ostomy effluent leakage, and the seal formed between the skin barrier material and the user may weaken. Oftentimes, the user may be unaware of, or cannot easily assess, the extent of weakening in the seal. Thus, the user may not become aware of a weakened seal, and consequently, the ostomy effluent may leak through to an exterior of the ostomy appliance. An ostomy leakage detection system may be used to detect such a leak. Such ostomy leakage detection systems can include subsystems that comprise a wearable device for alerting a user of an ostomy pouch system to the presence of ostomy effluent (or leakage) under a hydrocolloid skin barrier. Such a wearable device can connect to electronic sensors on the ostomy system to acquire and processes leak data. However, connecting to and securing the wearable device may be difficult for some users. For example, the wearable device may need a locking mechanism for opening and closing the wearable device with one hand.

Accordingly, it is desirable to provide an easy-to-use wearable device for ostomy leakage detection system.

### BRIEF SUMMARY

A wearable device for an ostomy leakage detection system is provided according to example embodiments.

In one aspect, an ostomy leakage detection system can include a wearable device and an adhesive patch. The wearable device may be configured to connect to a sensing accessory for detecting a leak. The wearable device may include a top housing, a bottom housing, and a hinge. The bottom housing may include a release layer. The adhesive patch may be configured to mount the wearable device. The adhesive patch may include a double-sided adhesive. The adhesive patch may include a first side configured to attach to a user and a second side configured to attach to the wearable device at the release layer.

In an embodiment, the release layer may include a low surface energy plastic.

In an embodiment, the adhesive patch may include a winged disk shape adhesive patch with at least one wing.

In an embodiment, the adhesive patch may include a kiss cut on the at least one wing for aiding in removing a release liner.

In an embodiment, the adhesive patch may include a nonwoven fabric with skin adhesive.

In an embodiment, the adhesive patch may include a double coated spunlace polyester nonwoven.

In an embodiment, the adhesive patch may include a single side silicone coated release liner with a kiss cut.

In an embodiment, the first side may include a biocompatible skin adhesive.

In an embodiment, the release layer is sized to fit on the first side of the adhesive patch without exposing an adhesive.

In an embodiment, the adhesive patch may include a winged disk-shaped double-sided adhesive.

In a second aspect, a wearable device can include a first housing, a second housing, a hinge, and a latching mechanism. The hinge may connect the first housing to the second housing. The latching mechanism may include a first latching member located on the first housing and a second latching member is spring activated and configured to receive the first latching member.

In an embodiment, the first housing may include a touch point. The second latching member may extend past an edge of the second housing. The wearable device may be configured for opening using a three-touchpoint design that allows for single handed opening.

In an embodiment, the second latching member may include a first and second end. The first end may be connected to the second housing at a hinge pin. The second end may be connected to a spring and configured to rotate at the hinge pin and within the second housing.

In an embodiment, the second latch member may include a latching area and a continuous perimeter wall configured for a maximum area to press against.

In an embodiment, the first latching member latches onto the second latching member within the latching area.

In an embodiment, the second housing may include a charging pad configured to make contact with a spring-loaded charging pin on a charging dock.

In an embodiment, the second housing may include a ramped protrusion for compressing the spring-loaded charging pin and providing a tactile and auditory notification.

In an embodiment, the second housing may include a wedge shape housing for maximum space savings.

In an embodiment, the second housing may include an alignment member that aligns the first and second housing, and the battery is positioned below the alignment member for space saving.

In an embodiment, the second housing may include light indicators configured to protrude out of the first housing.

The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1A is an illustration of an ostomy system according to an embodiment.

FIG. 1B is a front view of an ostomy system attached to a user, according to an embodiment.

FIG. 2 is a top-side perspective view of a wearable device in a closed position according to an embodiment.

FIG. 3 is a top-side perspective view of the wearable device of FIG. 2 in an open position.

FIG. 4 is a bottom-side perspective view of the wearable device of FIG. 2 in a closed position.

FIG. 5 is a top cross-section perspective view of the wearable device of FIG. 2 in an open position.

FIG. 6 is a cross-section view of the wearable device of FIG. 2 in a closed position.

FIG. 7 is a bottom perspective view of the wearable device of FIG. 2 and an adhesive patch according to an embodiment.

FIG. 8 is a top-side perspective view of the wearable device and the adhesive patch of FIG. 7.

FIG. 9 is a bottom-side perspective view of the wearable device and the adhesive patch of FIG. 7 in an attached position.

FIG. 10 is a front view of the wearable device and the adhesive patch of FIG. 7 attached to a user.

FIG. 11 is a schematic illustration of an adhesive patch according to an embodiment.

FIG. 12A is a front view of a plurality of adhesive patches provided in a sheet format according to an embodiment.

FIG. 12B is a rear view of the plurality of adhesive patches of FIG. 12A.

FIG. 13 is a front perspective view of the wearable device of FIG. 2 and an adhesive patch according to an embodiment.

FIG. 14A is a front cross-section view of the wearable device of FIG. 2 with a latching mechanism in an unlatched position.

FIG. 14B is a front cross-section view of the wearable device of FIG. 2 with a latching mechanism engaged but not yet in a fully latched position.

FIG. 14C is a front cross-section view of the wearable device of FIG. 2 with a latching mechanism in a latched position.

FIG. 15 is a top perspective view of a latching member of the wearable device of FIG. 2.

FIG. 16 is a bottom view of the wearable device of FIG. 2.

FIG. 17 is a top perspective view of the wearable device of FIG. 2 being opened by a user with one hand.

FIG. 18 is a side elevation view of the wearable device of FIG. 2.

FIG. 19 is a top-side perspective view of the wearable device of FIG. 2 and a charging dock according to an embodiment.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular. The words “first,” “second,” “third,” and the like may be used in the present disclosure to describe various information, such information should not be limited to these words. These words are only used to distinguish one category of information from another. The directional words “top,” “bottom,” up,“ ”down,“ front,” “back,” and the like are used for purposes of illustration and as such are not limiting. Depending on the context, the word “if” as used herein may be interpreted as “when” or “upon” or “in response to determining.”

The present disclosure provides a wearable device for an ostomy leakage detection system. The ostomy leakage detection system can be configured to detect ostomy effluent leakage under a skin barrier and to alert a user. The ostomy leakage detection system can provide multiple benefits to the user. For example, the system can allow the user to intervene and change a skin barrier and/or ostomy pouch system before a leak progresses which can cause embarrassment and inconvenience to the user. Further, the ostomy leakage detection system can assist in maintaining a user's skin health by detecting a leakage in its early stage to prevent prolonged skin exposure to ostomy effluent, which can lead to skin health complications. The ostomy leakage detection system can also support a user's emotional well-being by reducing anxiety associated with a risk of leakage. The ostomy leakage detection system may be applied to an ostomy barrier of a one-piece pouch system or a faceplate for a two-piece pouch system.

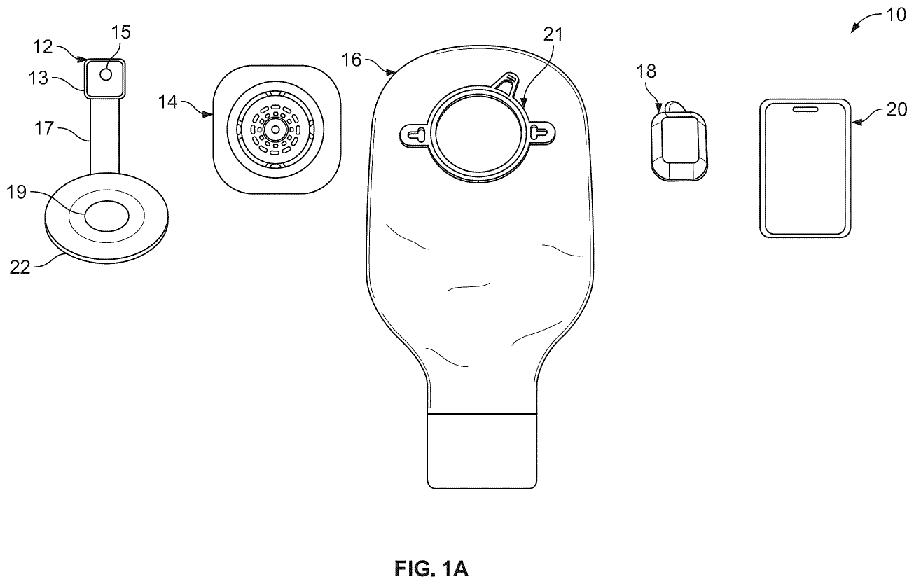

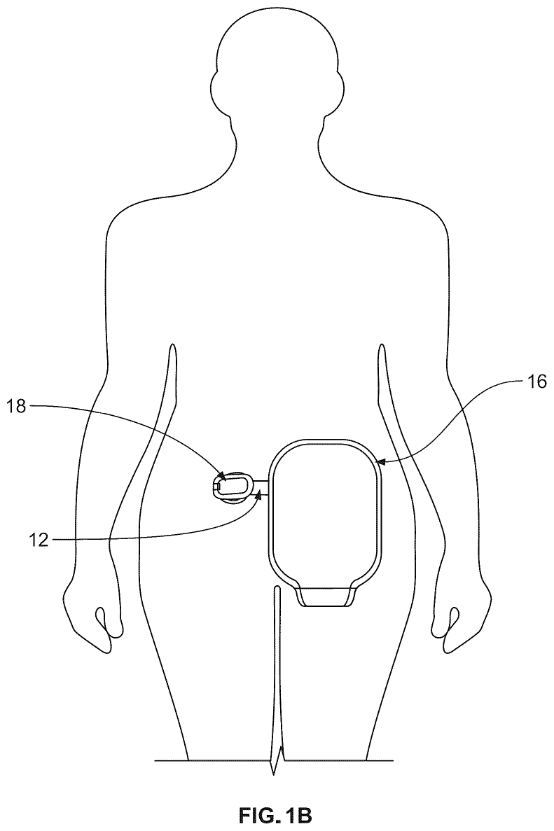

FIG. 1A illustrates an ostomy two-piece pouch system 10. According to example embodiments shown schematically in FIG. 1A, the ostomy system 10 can generally include a sensing accessory 12 with a tail end 13, an ostomy barrier appliance 14, an ostomy bag 16 with a pouch coupling member 21, a wearable device 18, and a mobile electronic device 20. The sensing accessory 12 can include the tail end 13 having a connector opening 15, a tail 17, a sensing ring 22 having an inlet opening 19.

FIG. 1B illustrates the ostomy pouch system 10 attached to a user according to example embodiments. As shown schematically in FIG. 1B, ostomy pouch system 10 can further include an adhesive (for example adhesive patch 110). According to example embodiments shown schematically in FIG. 1B, an end of the sensing accessory 12 can be attached to the user and can surround a stoma. The tail end 13 can be attached to the wearable device 18. The sensing accessory 12 can be designed to detect moisture around the stoma. The ostomy pouch 16 can be mounted on the ostomy barrier appliance 14 using the pouch coupling member 21. The wearable device 18 can be attached to the sensing accessory 12 using the connector opening 15. The wearable device 18 can be mounted on a user using a patch or an adhesive.

According to example embodiments, the ostomy leakage detection system may comprise three subsystems—the sensing accessory 12, the wearable device 18, and a mobile application on the mobile electronic device 20. The sensing accessory 12 may be provided as an accessory for an ostomy pouch system. The sensing accessory 12 can include sensors for detecting the presence of ostomy effluent. The sensing accessory 12 may be configured to communicate leakage detection signals to the wearable device 18.

### Wearable Device

The wearable device 18 may be configured to perform at least some processing of the leakage detection signals and alert a user of a leakage event. The wearable device 18 may be configured to electronically communicate with the mobile application through a wired or wireless communication system. Such electronic communications may include raw data acquired from the sensing accessory 12 or summarized leak data generated from processing the raw data. The wearable device 18 may also communicate system conditions, such as the presence of a sensing accessory, a faulty sensor, or a battery state. The mobile application may be a digital subsystem and/or software application installed and able run on the mobile electronic device 20. The mobile application may be configured to further process leak detection data and provide an alert or other information about an ostomy appliance to a user.

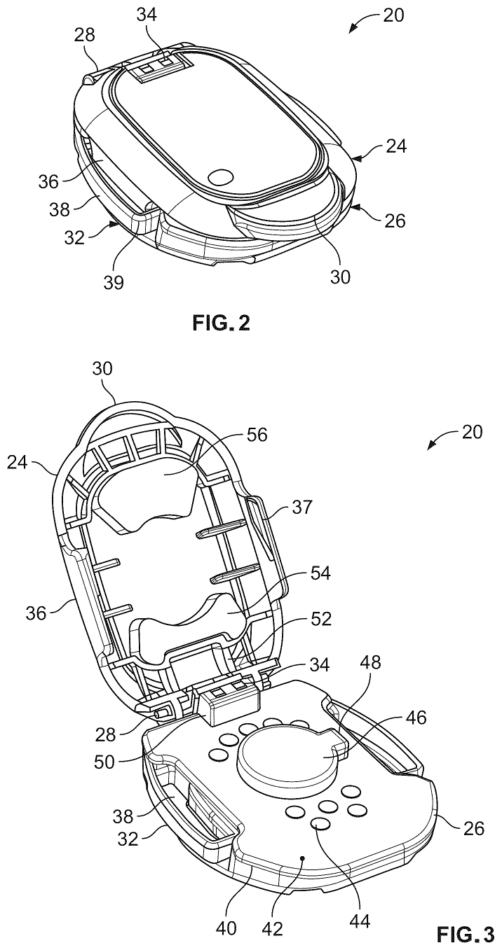

Referring back to the figures, FIGS. 2-10 and 13-19 show wearable device 18 according to an embodiment. The wearable device 18 may generally include a top housing 24, a bottom housing 26, a hinge 28, a lid member 30, a latching mechanism 32, and one or more light indicators 34.

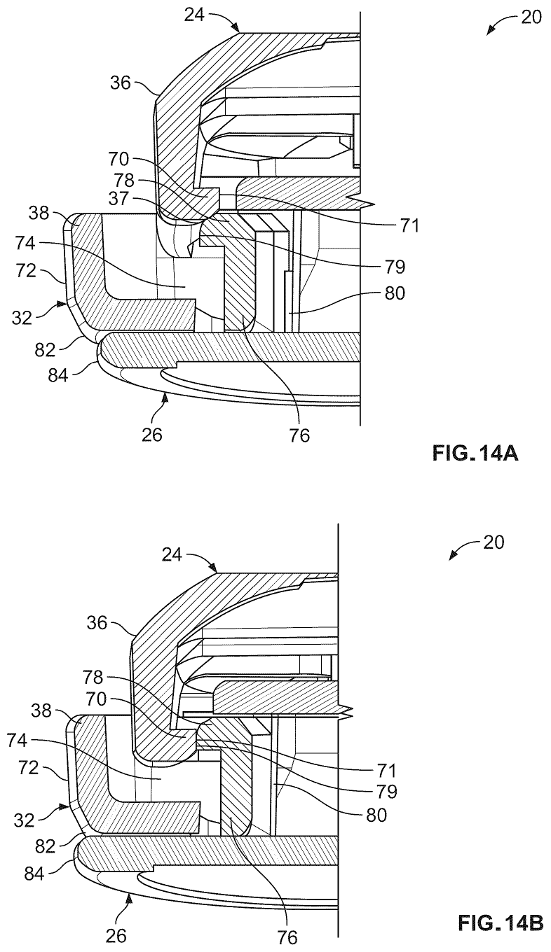

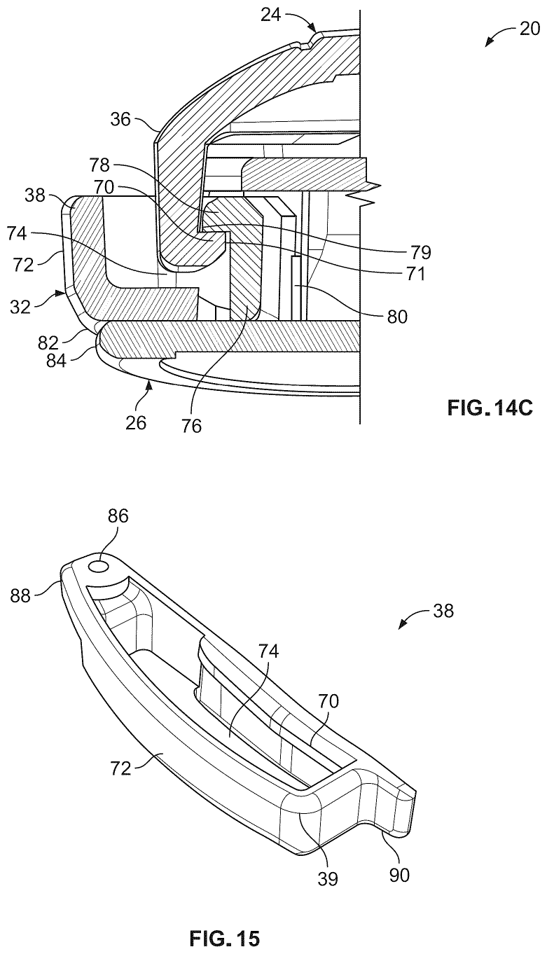

FIG. 2 shows a wearable device 18 in a closed position. The lid member 30 can be located on an edge of the top housing 24 and opposite the hinge 28. According to example embodiments shown schematically in FIG. 2, the lid member 30 can aid in opening and closing the wearable device 18. The lid member 30 may be a lid lift touch point. The latching mechanism 32 can include a latch member 36, a latch receiver 38, and an outer rotation end 39. The latch receiver 38 can receive the latch member 36 and secure the wearable device 18 in the closed position. The latch member 36 can include a protrusion member 37 (see FIG. 14A) that can engage with the latch receiver 38. The protrusion member 37 can protrude at one end of the latch member 36 and becomes narrower towards the center of the latch member 36. The outer rotation end 39 can rotate into the bottom housing 26 for operating the latching mechanism 32. The one or more light indicators 34 can be viewable from the top housing and configured to indicate conditions of wearable device 18 such a system status, battery status, and error messages.

FIG. 3 shows the wearable device 18 in an open position. According to example embodiments shown schematically in FIG. 3, the bottom housing 26 can include a sealed electronic enclosure 40, a pressure exchange vent 42, one or more sensor contact pads 44, a center raised member 46, a key member 48 and an alignment member 50. The alignment member 50 can include the one or more light indicators 34. The top housing 24 can include an alignment opening 52, and a first and second gasket 54, 56. The alignment member 50 can fit into alignment opening 52 for aligning the top housing 24 with the bottom housing 26 when closing wearable device 18. The one or more light indicators 34 can be located on top of the alignment member 50 and can protrude out of the top housing 24 for viewing when the wearable device 18 is closed. When wearable device 18 is closed, the first and second gaskets 54, 56 can press on the one or more sensor contact pads 44 and provide a water-tight seal. According to example embodiments, the first and second gaskets 54, 56 may be made out of a foam or elastomer material. As described more fully below, the center raised member 46 may act as a center alignment feature for the tail end 13 of sensing accessory 12. The alignment member 50 may be a peripheral raised member that can act as an alignment feature for the tail end 13 of sensing accessory 12 to align with the hinge for a correct connection between the sensing accessory 12 and the wearable device 18. The tail end 13 can have an alignment opening at its edge that can receive the alignment member 50 when the sensor accessory 12 is correctly connected to the wearable device 18.

According to example embodiments, the tail end 13 of sensing accessory can be physically and electronically coupled to the wearable device 18 by fitting tail end 13 over the interior face of the bottom housing 26. The tail end 13 can include a center opening, key opening, and alignment opening (not shown) which generally correspond in size, shape and orientation to center raised member 46, key member 48 and alignment member 50 along the interior face of the bottom housing 26. The tail end 13 may extend outward from and exit the wearable device 18 either in front towards the lid member 30 or on either of the sides adjacent or opposite latching mechanism 32. In any configuration, the center raised member 46, key member 48, and alignment member 50 can respectively align with the corresponding center opening, key opening, and alignment opening of tail end 13 and prevent the tail end 13 from being inserted backwards or upside down. The full alignment of all openings on the tail end 13 with their corresponding member on the interior face of the bottom housing ensure that the tail end 13 is in the correct position within the wearable device 18.

In another embodiment, the one or more light indicators 34 may be positoned on the center raised member 46 and configured to be viewed through one or more corresponding center openings on the top housing 24. In other embodiments, the one or more light indicators 34 may be located at other positions along the interior face of bottom housing 26 and configured to be viewed through one or more holes on the top housing 24.

According to example embodiments, the sensing accessory 12 can include one or more connection points (FIG. 1A). When the tail end 13 of sensing accessory 12 is secured within wearable device 18, the connection points can make contact with the one or more sensor contact pads 44 and provide an electrical connection between the sensing accessory 12 and the wearable device 18. Upon closing wearable device 18, the first and second gaskets 54, 56 along the interior face of top housing 24 can ensure contact between the one or more connections points and the one or more sensor contact pads 44 can backstop and press the corresponding members together. Gaskets 54, 56 can also provide a water-tight seal to prevent false leak detection.

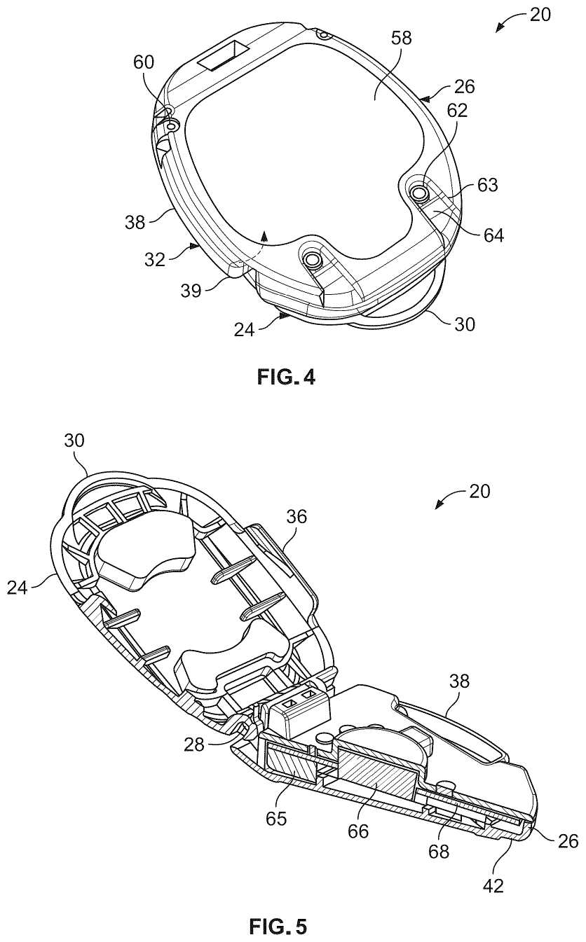

FIG. 4 shows the wearable device 18 in a closed position. According to example embodiments shown schematically in FIG. 4, the bottom housing 26 can include a release layer 58, a pin 60, charging pads 62, protrusions 64, and an opening 63. The release layer 58 can be located on the outer surface of the bottom housing 26 and configured to provide a flat surface for attaching the wearable device 18 to a user using an adhesive.

According to example embodiments, hinge pin 60 can connect the latch receiver 38 within the bottom housing 26. The latch receiver 38 can rotate at the outer rotation end 39 around the hinge pin 60 so that the receiver 38 can move into the bottom housing 26 for operating the latch mechanism 32.

Opening 63 can receive a charging pin 318 and ramped protrusion 319 of a charging dock 310 for charging (FIG. 19). The charging pads 62 can be conductive and positioned alongside and be electrically coupled to the conductive charging pins 318 for charging the wearable device 18. The opening 63 can align the wearable device 18 with the ramped protrusion 319 for facilitating a secure contact between the charging pins 318 and the charging pads 62. The one or more protrusions 64 can make contact with ramped protrusion 319 of the charging dock and provide tactile feedback to a user when the wearable device 18 is being inserted into the charging dock 310.

FIG. 5 shows a top perspective cross-section view of the wearable device 18 in an open position. According to example embodiments shown schematically in FIG. 5, the wearable device 18 can include a motor 65, a battery 66, and a printed circuit board (PCB) 68 within the sealed electronic enclosure 40. The motor 65 can provide a haptic buzzer for silent notifications. The battery 66 may be aligned within the center raised member 46 for space savings. The PCB 68 can include a processing unit, a communication unit, and electronic connectors that connect with the one or more sensor contact pads 44. The PCB 68 can analyze signals received from the sensing accessory 12, communicate with a mobile device 20 or charging dock 310, and alert a user through sound and vibration, as well as one or more lights to indicate a system status.

In alternate embodiments, the motor 65 may be positioned within the center raised member 46, and the battery 66 may be positioned below the alignment member 50.

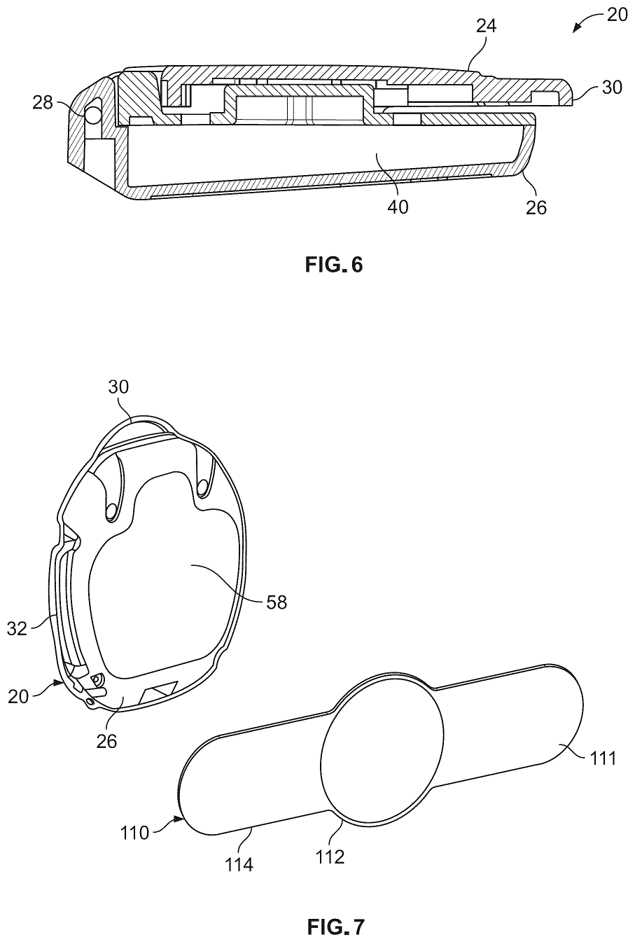

FIG. 6 is a cross-section view of the wearable device 18 in a closed position. According to example embodiments shown schematically in FIG. 6, the wearable device 18 can include a sealed electronic enclosure 40 in a wedge shape for maximum space savings.

### Adhesive Patch

According to example embodiments, the ostomy pouch system 10 can include an adhesive patch 110 for mounting the wearable device 18 thereto. The adhesive patch 110 can be a thin engagement mechanism, of low cost, that may be easily modified.

FIG. 7 shows the wearable device 18 and a representative adhesive patch 110 according to embodiments presented herein. According to example embodiments shown schematically in FIG. 7, the adhesive patch 110 can be a winged disk-shaped double-sided adhesive patch. The adhesive patch 110 can include a body-side 111, a center area 112 and one or more wings 114. The center area 112 may be configured to attach, on a distal side 113, to the release layer 58 of the wearable device 18. The one or more wings 114 can provide an area, on the body-side 111, to attach to the user's skin. The winged shape of wings 114 allows for the wearer to remove the adhesive patch with the wearable device 18 still attached. The adhesive patch 110 can then be removed from the wearable device 18. Alternatively, the wings 114 can be used to hold the adhesive patch 110 on the user while removing the wearable device 18 from the adhesive patch 110.

According to example embodiments, the center area 112 may be of a disk or circular shape. In other embodiments, the center area 112 may be of a square shape (FIG. 13), a diamond or any other shape without limitation.

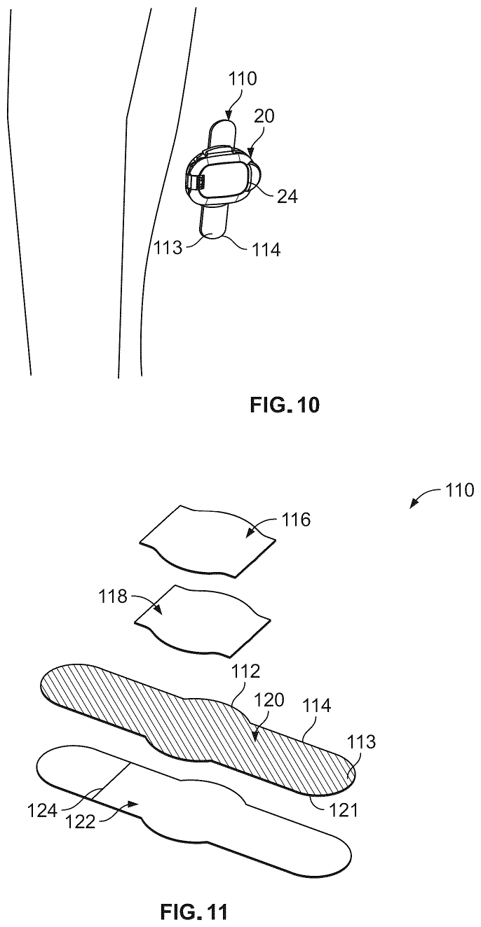

According to example embodiments, the adhesive patch 110 may be mounted to the abdomen of a user (FIG. 10). In other embodiments, the adhesive patch 110 may be mounted on the ostomy bag 16.

According to example embodiments, the release layer 58 can include a low surface energy plastic which can allow for an easy and clean release from the adhesive patch 110. One major drawback of using an adhesive to mount a wearable device can be that an adhesive residue may remain attached to the wearable device after removal from the adhesive. A buildup of adhesive residue could potentially interfere with the function of the wearable device, or function of a charger or charging dock. The low surface energy plastic prevents adhesive buildup on the wearable device from the repeated application and removal of the device from the adhesive patches by ensuring that the adhesive pulls away from the wearable device completely.

According to example embodiments, a low surface energy overlaminate can be applied to the wearable device 18 in the release layer 58. In another embodiment, a similar design can be achieved by molding the release layer 58 into the low surface energy plastic itself.

The low surface energy plastic may include a polyethylene overlaminate, which may be a part of a label construction. Such a laminate can protect the label from being damaged from the double-sided tape, while providing appropriate release values for the double-sided tape. The release value can be balanced such that the wearable device is securely attached to the double-sided tape during use but releases cleanly from the double-sided tape to prevent any residue build up or damage to the wearable device.

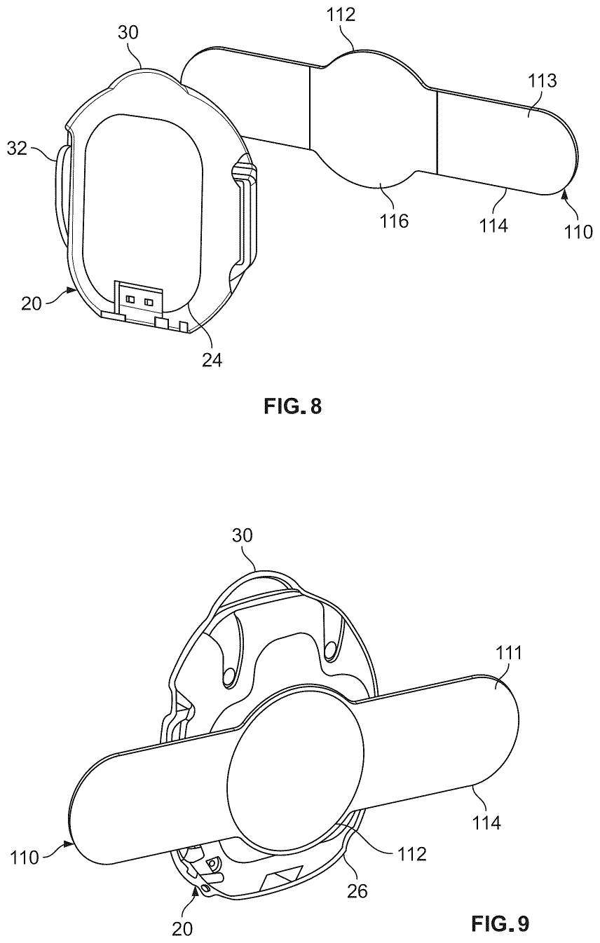

FIG. 8 shows the wearable device 18 and the adhesive patch 110. According to example embodiments shown schematically in FIG. 8, the adhesive patch 110 can include a distal side 113, and a first release liner 116.

FIG. 9 shows the wearable device 18 and the adhesive patch 110 in an attached position. According to example embodiments shown schematically in FIG. 9, the release layer 58 of the wearable device 18 can be covered by the center area 112. The lid member 30 can be perpendicular to the wing 114. In another embodiment, the lid member 30 may be parallel to the wing 114 (FIG. 13).

FIG. 10 shows the wearable device 18 mounted on a user using the adhesive patch 110. According to example embodiments shown schematically in FIG. 10, the wearable device 18 can be attached on the adhesive patch 110 and the adhesive patch 110 can be secured to the abdomen of a user. The one or more wings 114 can be accessible to the user for removing the adhesive patch 110 without removing the wearable device 18 from the patch.

FIG. 11 shows a representative construction of an adhesive patch 110 according to an example embodiment. According to example embodiments shown schematically in FIG. 11, the adhesive patch 110 can include the first release liner 116, an adhesive layer 118, a nonwoven layer 120, a skin adhesive layer 121 on the body side 111, and a second release liner 122. The second release liner 122 can include a kiss cut 124. The second release liner 122 may be removed from the distal side 111 of the nonwoven layer 120 at the kiss cut 124.

The adhesive layer 118 can be provided on a selective area opposite the skin adhesive layer 121 to adhere to the release layer 58. The release layer 58 and adhesive layer 118 can be configured such that there is no exposed adhesive after the user places the device on their skin using the adhesive patch 110 (FIGS. 9 and 10). The adhesive layer 118 may be a double coated spunlace polyester nonwoven layer. The nonwoven layer 120 may be a low-density polyethylene (LDPE) nonwoven fabric with skin adhesive. The skin adhesive 121 may be a biocompatible skin adhesive.

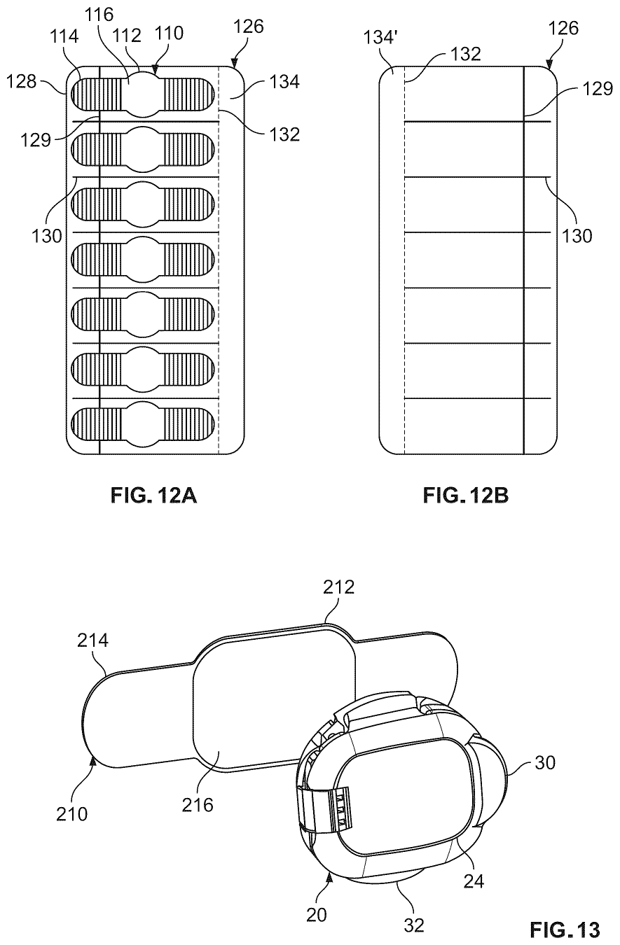

FIG. 12A shows a front view of an adhesive patch sheet 126 with multiple adhesive patches 110 according to an embodiment. According to example embodiments shown schematically in FIG. 12A, the adhesive patch sheet 126 can include a release liner 128 with a kiss cut 129, a selective thru slit 130, a perforation 132, and a main section 134. The adhesive patch sheet 126 can be a single-side silicon coated release liner that can be used to hold the adhesive patches 110 for use. Each individual adhesive patch 110 may be removed from the adhesive patch sheet 126 for individual use. The selective thru slit 130 can prevent adjacent adhesive patches from moving independently of each other and to separate an individual adhesive patch 110 from the other adhesive patches 110. The perforation 132 can separate an individual adhesive patch 110 from the adhesive patch sheet 126. The main section 134 can hold the adhesive patches. The main section 134 may be used to attach the adhesive patch sheet 126 to corresponding packaging so that each individual release liner 128 may be removed while the adhesive patch sheet 126 stays on the packaging. The kiss cut 124, 129 may aid in releasing the release liner 122, 128. The kiss cut 124, 129 can be on the wing 114 so when the adhesive patch 110 is first attached to wearable device 18, the wing 114 can flex to remove the release liner 122, 128.

FIG. 12B is a back view of the adhesive patches 110 provided in the sheet format. According to example embodiments shown schematically in FIG. 12B, the adhesive patch sheet 126 can include a back side of the main section 134′. The adhesive patch sheet 126 can be attached to corresponding packaging using the main section back side 134′. The adhesive patch sheet 126 can also be attached to another adhesive patch sheet 126 using the main section back side 134′.

FIG. 13 shows a front side view of the wearable device 18 and an adhesive patch 210 according to another embodiment. According to example embodiments shown schematically in FIG. 13, the adhesive patch 210 can include a center area 212, a wing 214, and an adhesive layer 216. The wearable device 18 can be attached on the center area 212 and align the lid member 30 with the wing 214.

### Mechanical Latch

According to example embodiments provided herein, the wearable device 18 can include a closure mechanism used to keep the wearable device closed and secured. The closure mechanism may comprise latching mechanism 32 comprising a latch member 36 on the top housing 24 of the wearable device, and an interfering member latch receiver 38 on the bottom housing 26 of the wearable device. The latch member 36 can be held in place until the interfering member latch receiver 38 can be moved out of the way, and the latch member 36 can be released to move freely relative the receiver. This type of closure mechanism allows a force to press on the tail end 13 against the contact pads 44 of the wearable device 18 (FIG. 14C).

The latch mechanism 32 allows the wearable device 18 to have a high holding force to resist the opening force the elastomer pad imparts on the lid while pressing the tail end 13 against the contact pads 44, but still allows for easy opening of the top housing 24 as needed.

According to example embodiments, the latch mechanism 32 can be engaged by pushing the latch receiver 38 into the bottom housing 26 and out of the way and a spring (not shown) which can be compressed when the wearable device 18 lid is pressed closed. Once opposing surfaces 71, 79 of the latching member and receiver 36, 38 have passed each other, a spring force pushes the latch receiver 38 away from the bottom housing 26 to engage the latch member 36. The latching faces of the different components can be flush against and parallel to each other so that the lid remains locked in place until the mechanism is depressed to release the lid.

According to example embodiments, the latch receiver 38 may be of a different color than the rest of the wearable device 18. For example, the wearable device 18 can be a light gray or white color and the latch receiver 38 can be a dark grey color. This may be done to draw the user's attention to the separate members of the latch mechanism for improved identification of the latch member 36 as being the feature that is to be depressed to allow for the engagement and disengagement of the latch mechanism and the corresponding opening and securing of the module.

The latch mechanism 32 can securely close the wearable device 18 and easily open the wearable device 18. The latch mechanism 32 may have three positions or states. An open state, an engaging state, and a latched state.

FIG. 14A shows a cross-section view of the wearable device 18 with the latching mechanism 32 in an open state. According to example embodiments shown schematically in FIG. 14A, the latch member 36 can include an L-shaped member 70 with a surface 71. The L-shaped member 70 can include the protrusion member 37. The latch receiver 38 can include a continuous perimeter wall 72, a pass-through core 74, a lower latch member 76 comprising an inverted L-shaped member 78 with a surface 79, and a bottom housing opening 80. The latch receiver 38 can include a latch receiver edge 82. The wearable device 18 can include a bottom housing edge 84.

As shown schematically in FI. 14A, in the open state, the latch member 36 can be positioned above and outside of the latch receiver 38. The latch receiver 38 can include a spring (not shown) to keep the latch receiver edge 82 extended past the bottom housing edge 84 and the bottom housing opening 80.

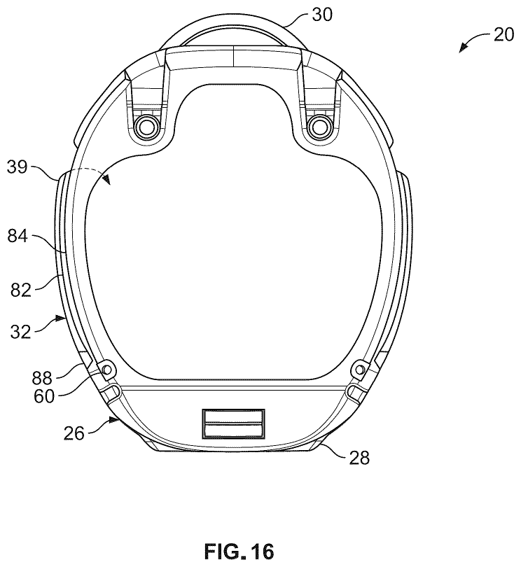

FIG. 14B shows a cross-section view of the wearable device 18 with the latching mechanism 32 in an engaging state. According to example embodiments shown schematically in FIG. 14B, in the engaging state the inverted L-shaped member 78 can rotate around the pin 60 (See FIGS. 4 and 16) and into the bottom housing 26. The rotation allows the L-shaped member 70 of latch member 36 to move into the pass-through core 74 and begin to engage the inverted L-shaped member 78. The rotation can actuate the bottom housing edge 84 to extend past the latch receiver edge 82 and the bottom housing opening 80 to narrow.

The engaging state can be activated by the top housing 24 being pushed onto the bottom housing 26. The force can move the L-shaped member 70 into the pass-through core 74 and move the inverted L-shaped member 78 towards the opening 80 by inducing the latch receiver 38 to rotate around the hinge pin 60.

The engaging state can also be activated by a user pressing on the continuous perimeter wall 72 towards the opening 80 and causing the latch receiver 38 to rotate around the hinge pin 60 into the bottom housing 26. This rotation may allow the latch member 36 to move into the pass-through core 74.

FIG. 14C is a cross-section view of the wearable device 18 with the latching mechanism 32 in a latched state. According to example embodiments shown schematically in FIG. 14C, in the latched state the upward-facing surface of L-shaped member 70 can engaged with the downward-facing surface of inverted L-shaped member 78 in the pass-through core 74. In such state, the latch receiver 38 can be rotated away from the bottom housing 26 to enable the latch receiver edge 82 to extend past the bottom housing edge 84 and the housing opening 80 to widen.

In this latched state, the closure mechanism allows a force between the top and bottom housing 24, 26 to press on the tail end 13 against the contact pads 44 of the wearable device 18. This force is independent of the force needed to close or open the wearable device 18 since the force is created when the latch member 36 is latched to the latch receiver 38 as the top and bottom housing 24, 26 pull on each other through the latch member 36 and latch receiver 38.

From the latched state, the latching mechanism 32 can be opened by pushing inward on the continuous perimeter wall 72, which can induce the inverted L-shaped member 70 to rotate towards the opening 80 and allow the L-shaped member 78 to be moved out of the pass-through core 74. The L-shaped member 78 may be moved out of the pass-through core 74 by lifting the lid member 30 away from the bottom housing 26.

In addition to the usability benefit, the latches can be positioned directly adjacent to the area where the tail end 13 of sensing accessory 12 makes contact with the contact pads 44. The adjacent location of the latches 36, 38 is intentional so that the flexure of the housing lid can be minimized by having the latching point as close to the center of the contact zone where the connection points of the sensing accessory 12 make contact with the contact pads 44 as possible.

The shape of the latches 36, 38 can be designed to be as small as possible while creating the maximum amount of surface area to press against as to not create discomfort during actuation. To achieve this, the latch receiver 38 with a pass-through core 74 can be produced by way of an injection mold tool to create the latching area. This enables a part design that has the continuous perimeter wall 72 with no need for breaks or low points in the plastic.

FIG. 15 shows the latch receiver 38 of the wearable device. According to example embodiments shown schematically in FIG. 15, the latch receiver 38 can include a first end 88 and second end 90. The first end 88 can include a pivot point 86 that can connect to the bottom housing 26 at pin 60. The second end 90 can be located and rotate within the bottom housing 26.

FIG. 16 shows the wearable device 18 in a latched state. According to example embodiments shown schematically in FIG. 16, the latch receiver edge 82 can extend past the bottom housing edge 84 when the latching mechanism is in the open state and latched state. The outer rotation end 39 can rotate on the pin 60 into the bottom housing 26 in the engaging state.

The latch receiver 38 can follow the profile of the bottom housing 26 but can extend slightly outside of the profile to allow easy access, but prevent accidental actuation during wear.

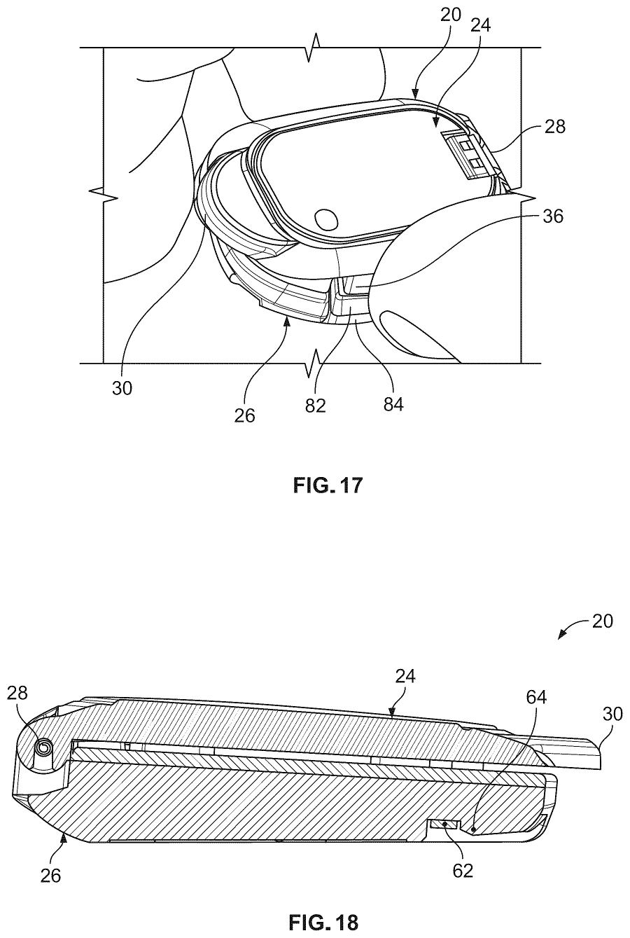

FIG. 17 shows the wearable device 18 being opened by a user with one hand. According to example embodiments shown schematically in FIG. 17, the wearable device 18 can use a three-touchpoint design that can allow for single handed opening. The two latches on either side of the wearable device 18 can be pinched together using opposing force from two fingers, and a third finger can lift the top housing 24. When the sensor accessory 12 is connected to the wearable device 18, the three-touchpoint design can still be accessible so the wearable device 18 can be opened in the same way.

In another embodiment, the closure mechanism may be a snap-fit mechanism, where a deformable structure mates with another structure of the wearable device to keep the device closed. In this type of mechanism, the structure elastically deforms when under load to allow for movement between the latched and unlatched states.

### Charging Components

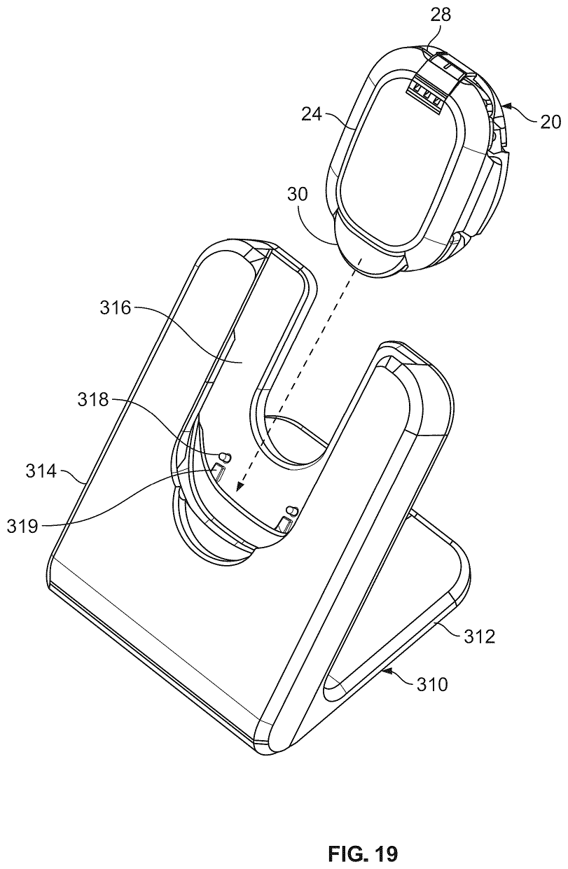

FIG. 18 shows a cross-section view of the wearable device 18. According to example embodiments shown schematically in FIG. 18, the protrusions 64 can include a peak-like shape for engaging a spring loaded pin 318 (FIG. 19). The charging pads 62 can be located within the wearable device 18 and below the protrusions 64.

FIG. 19 shows a top side view of the wearable device 18 and a charging dock 310 according to an embodiment. As shown schematically in FIG. 19, the charging dock 310 can include a base 312 and a top section 314 with an opening 316. The opening 316 can include one or more spring-loaded pins 318.

The charging dock 310 can include a slide-in mechanism configured to receive the wearable device 18 for charging. The ramped protrusions 319 can align the wearable device 18 for charging.

The wearable device 18 can use a snap positioning mechanism for tactile/auditory notification of correct placement in the charging dock 310. The function of the snap positioning mechanism itself can be split between the wearable device 18 and the charging dock 310. The wearable device 18 can have charging pads 62 on its bottom housing 26 that engage spring loaded pins 318 within the charging dock 310. The ramped protrusion 319 can be in front of the charging pad 62. When the wearable device 18 is pushed into the charging dock 310, the spring-loaded pins 318 can engage the protrusions 64 and become compressed until the user provides enough force to overcome the peak of the protrusion. After the peak shape or ramp, the protrusions can slope down and allow the spring-loaded pins 318 to engage the charging pads 62. The charging dock 310 housing can prevent any further forward movement of the wearable device 18 to ensure correct location. This snap action combined with the hard stop against the charger housing creates the tactile and auditory feedback that the wearable device 18 is positioned correctly in the charging dock.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. An ostomy leakage detection system comprising:
a wearable device configured to connect to a sensing accessory for detecting a leak, wherein the wearable device comprises a top housing, a bottom housing, and a hinge, wherein the bottom housing comprises a release layer; and
an adhesive patch configured to mount the wearable device, wherein the adhesive patch comprises a double-sided adhesive, wherein the adhesive patch comprises a first side configured to attach to a user and a second side configured to attach to the wearable device at the release layer.

2. The ostomy leakage detection system of claim 1, wherein the release layer comprises a low surface energy plastic.

3. The ostomy leakage detection system of claim 1, wherein the adhesive patch comprises a winged disk shape adhesive patch with at least one wing.

4. The ostomy leakage detection system of claim 3, wherein the adhesive patch comprises a kiss cut on the at least one wing for aiding in removal of a release liner.

5. The ostomy leakage detection system of claim 1, wherein the adhesive patch comprises a nonwoven fabric with skin adhesive.

6. The ostomy leakage detection system of claim 1, wherein the adhesive patch comprises a double coated spunlace polyester nonwoven.

7. The ostomy leakage detection system of claim 1, wherein the adhesive patch comprises a single side silicone coated release liner with a kiss cut.

8. The ostomy leakage detection system of claim 1, wherein the first side comprises a biocompatible skin adhesive.

9. The ostomy leakage detection system of claim 1, wherein the release layer is sized to fit on the first side of the adhesive patch without exposing an adhesive.

10. The ostomy leakage detection system of claim 1, wherein the adhesive patch comprises a winged disk-shaped double-sided adhesive.

11. A wearable device comprising:
a first housing;
a second housing;
a hinge that connects the first housing to the second housing; and
a latching mechanism, wherein the latching mechanism comprises a first latching member located on the first housing and a second latching member located on the second housing, wherein the second latching member is spring activated and configured to receive the first latching member.

12. The wearable device of claim 11, wherein the second latching member extends past an edge of the second housing, and wherein the wearable device is configured for opening using a three-touchpoint design that allows for single handed opening.

13. The wearable device of claim 11, wherein the second latching member comprises a first and second end, wherein the first end is connected to the second housing at a hinge pin, and wherein the second end is connected to a spring and configured to rotate at the hinge pin and within the second housing.

14. The wearable device of claim 12, wherein the second latch member comprises a latching area and a continuous perimeter wall configured for a maximum area to press against.

15. The wearable device of claim 14, wherein the first latching member latches onto the second latching member within the latching area.

16. The wearable device of claim 1, wherein the second housing comprises a charging pad configured to make contact with a spring-loaded charging pin on a charging dock.

17. The wearable device of claim 16, wherein the second housing comprises a ramped protrusion for compressing the spring-loaded charging pin and providing a tactile and auditory notification.

18. The wearable device of claim 12, wherein the second housing comprises contact pads for connecting to a sensing accessory, and wherein the latching mechanism is located near the contact pads.

19. The wearable device of claim 11, wherein the second housing comprises a wedge shape housing for maximum space savings.

20. The wearable device of claim 11, wherein the second housing comprises an alignment member that aligns the first and second housing, and wherein a battery is positioned below the alignment member for space saving.
